(19) United States
(12) Patent Application Publication
(10) Pub. No.: US 2002/0193872 A1
(43) Pub. Date: Dec. 19, 2002

(54) Prosthetic graft assembly and method of use

(76) Inventors: Hugh H. Trout III, Washington, DC (US); Howard M. Tanner, Logan, UT (US)

Correspondence Address: COLLIER, SHANNON, SCOTT, PLLC, 3050 K STREET, NW, SUITE 400, WASHINGTON, DC, 20007, US

(21) Appl. No.: 10/173,028

(22) Filed: Jun. 18, 2002

Related U.S. Application Data
(60) Provisional application No. 60/298,410, filed on Jun. 18, 2001.

(51) Int. Cl. A61F 2/06

(52) U.S. Cl. 623/1.34; 623/1.35

(57) ABSTRACT

The present invention relates generally to a surgical component and a surgical component assembly, such as a prosthetic graft, and methods of use. In particular, embodiments of the present invention relate to a surgical component and surgical component assembly for use during a surgical procedure for repairing a vessel. The surgical component in accordance with the present invention may comprise a body portion having at least one orifice located thereon. The body portion and orifice(s) may be customized to an individual surgical patient and may further be marked by indicators for use during a surgical procedure.

### CROSS-REFERENCE TO RELATED APPLICATIONS

The present invention relates to, and is entitled to the benefit of the earlier filing date and priority of, U.S. Provisional Application Serial No. 60/298,410, filed Jun. 18, 2001.

### FIELD OF THE INVENTION

The present invention relates generally to a surgical component, such as a prosthetic graft, and a surgical component assembly and methods of use. In particular, the present invention relates to a surgical component and surgical component assembly for use during a surgical procedure. Embodiments of the present invention are directed to a prosthetic graft and a prosthetic graft assembly for use in repairing a vessel during a surgical procedure.

### BACKGROUND OF THE INVENTION

An aneurysm is a ballooning of the wall of an artery resulting from the weakening of the artery due to disease or other conditions. Left untreated, the aneurysm will frequently rupture, resulting in loss of blood through the rupture and death.

Aortic aneurysms are the most frequent form of arterial aneurysm and are life threatening. The aorta is the main artery which supplies blood to the circulatory system. The aorta arises from the left ventricle of the heart, passes upward and bends over behind the heart, and passes down through the thorax and abdomen. Among other arterial vessels branching off the aorta along its path, the abdominal aorta supplies two side vessels to the kidneys, the renal arteries. Below the level of the renal arteries, the abdominal aorta continues to about the level of the fourth lumbar vertebrae (or the navel), where it divides into the iliac arteries. The iliac arteries, in turn, supply blood to the lower extremities and perineal region.

It is common for an aortic aneurysm to occur in that portion of the abdominal aorta between the renal arteries and the iliac arteries. This portion of the abdominal aorta is particularly susceptible to weakening, resulting in an aortic aneurysm. Such an aneurysm is often located near the iliac arteries. An aortic aneurysm larger than about 5 cm in diameter in this section of the aorta is ominous. Left untreated, the aneurysm may rupture, resulting in rapid, and usually fatal, hemorrhaging. Typically, a surgical procedure is not performed on aneurysms smaller than 5 cm because presently no statistical benefit exists in performing such procedures.

Aneurysms in the abdominal aorta are associated with a particularly high mortality rate; accordingly, current medical standards call for urgent operative repair. Abdominal surgery, however, results in substantial stress to the body. Although the mortality rate for an aortic aneurysm is extremely high, there is also considerable mortality and morbidity associated with open surgical intervention to repair an aortic aneurysm. This intervention involves penetrating the abdominal wall to the location of the aneurysm to reinforce or replace the diseased section of the aortic aneurysm. A prosthetic device, typically a synthetic tube graft, is used for this purpose. The graft serves to exclude the aneurysm from the circulatory system, thus relieving pressure and stress on the weakened section of the aorta at the aneurysm.

Repair of an aortic aneurysm by surgical means is a major operative procedure. Substantial morbidity accompanies the procedure, resulting in a protracted recovery period. Further, the procedure entails a substantial risk of mortality. While surgical intervention may be indicated and the surgery carries attendant risk, certain patients may not be able to tolerate the stress of intra-abdominal surgery. It is, therefore, desirable to reduce the mortality and morbidity associated with intra-abdominal surgical intervention.

In recent years, apparatus and methods have been developed attempting to treat an aortic aneurysm without the attendant risks of intra-abdominal surgical intervention. Among them are inventions disclosed and claimed to endovascular and endoluminal prosthetics. The prosthetics appear to be promising but are significantly restricted. Primarily, current prosthetics are limited by the prosthetic's degree of similarity to the diseased vascular or luminal vessel the prosthesis will be used to repair. The size, location, position, and specific geometry of the aortic aneurysm, however, is dependent on an individual patient and very often aortic aneurysms occur in close proximity to, if not along, lateral branching vessels. The specific geometry between the abdominal aorta and iliac arteries, and extent of disease infestation, and the integrity of the vessel wall are often determinant factors in the availability of use of known prosthetics.

Additionally, in order to restore a vessel wall, the prosthesis must radiate within the diseased wall producing a fairly convoluted route. Not only does the prosthesis need to reach into various branching diseased vessels but must also seal and infuse itself to a healthy vessel wall.

Currently known branching endovascular and endoluminal prosthetics are formed as tubular, radially expandable stent-grafts. A separate graft is normally appended to the cylindrical frame of the vessel wall reducing the incidence of blood flow through a ruptured vessel wall. The stent-grafts are often made of inflexible fabrics to prevent pressure from enlarging a weakened vascular or luminal wall. Thus, the resulting stent-graft comprises a uniform structure with smaller iliac branch points parallel to the aortic portion.

Typically these stent-grafts are anticipated to undergo minimal contortions accommodating the various branch angles of the body system. Often, these impositions result in folding, kinking, or wrinkling of the graft. Thus, the stent-graft occludes instead of adding therapeutic value. Even if a full occlusion does not occur, the anticipated torsion of the stent-graft branch points may become unbalanced leading to flow induced pressure further increasing the risk of rupture. Typically, stent-grafts, because of these limitations, are only available to a narrow set of aneurysm patients.

What is needed, therefore, is a variable graft attachment that minimizes the disruption of blood flow to branching vessels. The variable graft attachment should be disposed on a tubular or bifurcated graft with an orifice accommodating the various locations of branching vessels. Additionally, the variable graft attachment should accommodate the position and number of branching vessels and complement the size, shape, length, and width of the orifice in the tubular or bifurcated graft specific to a surgical patient's anatomy.

It is therefore an advantage of some, but not necessarily all, embodiments of the present invention to provide a surgical component that minimizes the disruption of blood flow to branching vessels.

It is another advantage of embodiments of the present invention to provide a surgical component having at least one orifice located thereon for accommodating branching vessels and permitting continuous blood flow.

It is yet another advantage of embodiments of the present invention to provide a surgical component that may be customized to an individual surgical patient.

Additional advantages of various embodiments of the invention are set forth, in part, in the description that follows and, in part, will be apparent to one of ordinary skill in the art from the description and/or from the practice of the invention.

### SUMMARY OF THE INVENTION

Responsive to the foregoing challenges, Applicant has developed an innovative surgical component for use during a surgical procedure. An embodiment of the surgical component comprises: an elongated body having an open proximal end and an open distal end, wherein the body further comprises at least one orifice located between the proximal end and the distal end. The elongated body may be customized to a surgical patient.

The at least one orifice may be customized for use during the surgical procedure. The surgical component may further comprise an indicator complementary to the at least one orifice, wherein the at least one orifice is delineated by the indicator. The indicator may comprise a plurality of radiopaque markers. The surgical component may further comprise an indicator complementary to the elongated body, wherein the elongated body is delineated by the indicator. The indicator may comprise a plurality of radiopaque markers.

According to another embodiment of the present invention, the surgical component for use during a surgical procedure comprises: an elongated body having an open proximal end and an open distal end and one or more limbs attached thereto, wherein the body further comprises at least one orifice located between the proximal end and the distal end. The elongated body may be customized to a surgical patient.

The at least one orifice may be customized for use during the surgical procedure. The surgical component may further comprise an indicator complementary to the at least one orifice, wherein the at least one orifice is delineated by the indicator. The indicator may comprise a plurality of radiopaque markers. The surgical component may further comprise an indicator complementary to the elongated body and the one or more limbs, wherein the body and the limbs are delineated by the indicator. The indicator may comprise a plurality of radiopaque markers.

ln another embodiment, the surgical component for use during a surgical procedure comprises a body portion having at least one orifice located thereon. The body portion may be flat. The body portion may be customized for use during the surgical procedure.

According to this embodiment, the at least one orifice may be customized for use during the surgical procedure. The surgical component may further comprise an indicator complementary to the at least one orifice, wherein the indicator delineates the at least one orifice. The indicator may comprise a plurality of radiopaque markers. The surgical component may further comprise an indicator complementary to the body portion, wherein the indicator delineates the body portion. The indicator may comprise a plurality of radiopaque markers.

In another embodiment of the present invention, a surgical component assembly for use during a surgical procedure comprises: a first surgical component comprising an elongated body with an open proximal end and an open distal end, wherein the first surgical component further comprises at least one orifice located between the proximal end and the distal end; and a second surgical component comprising a body portion adapted for placement over at least a portion of the at least one orifice of the first surgical component.

The body portion of the second surgical component may further comprise at least one orifice located thereon. The at least one orifice of the second surgical component may be of a smaller diameter than the at least one orifice of the first surgical component.

The surgical component assembly may further comprise a first indicator complementary to the at least one orifice of the first surgical component and a second indicator complementary to the at least one orifice of the second surgical component for delineating the orifices. The first indicator and the second indicator may be the same, or different. The surgical component assembly may further comprise a first indicator complementary to the elongated body of the first surgical component and a second indicator complementary to the body portion of the second surgical component for delineating the first surgical component and the second surgical component.

According to an embodiment of the surgical component assembly, the first surgical component may further comprise one or more limbs attached to the elongated body. The body portion of the second surgical component may further comprise at least one orifice located thereon. The at least one orifice of the second surgical component may be of a smaller diameter than the at least one orifice of the first surgical component. The surgical component assembly may further comprise a first indicator complementary to the at least one orifice of the first surgical component and a second indicator complementary to the at least one orifice of the second surgical component for delineating the orifices. The first indicator and the second indicator may be the same, or different. The surgical component assembly may further comprise a first indicator complementary to the elongated body and the one or more limbs of the first surgical component and a second indicator complementary to the body portion of the second surgical component for delineating the first surgical component and the second surgical component.

The present invention is also directed to a method of customizing a surgical component for use in a surgical patient, comprising the step of: adjusting the size of the surgical component to conform to the surgical patient. The method may further comprise one or more of the steps of: adjusting the position of at least one orifice located on the surgical component; adjusting the shape of at least one orifice located on the surgical component; and adjusting the number of orifices located on the surgical component.

The present invention is further directed to a method of labeling a surgical component having at least one orifice for use in a surgical procedure, comprising the step of: radiopaquing the at least one orifice such that the at least one orifice is identifiable.

The present invention is also directed to a method of labeling a surgical component for use in a surgical procedure, comprising the step of: radiopaquing a body portion of the surgical component such that the surgical component is identifiable.

The present invention is also directed to a method of repairing a vessel at a surgical site, comprising the steps of: delivering a first surgical component having at least one orifice thereon to the surgical site; positioning the at least one orifice of the first surgical component to correspond to at least one branching vessel; and positioning a second surgical component over at least a portion of the least one orifice of the first surgical component.

It is to be understood that both the foregoing general description and the following detailed description are exemplary and explanatory only, and are not restrictive of the invention, as claimed. The accompanying drawings, which are incorporated herein by reference, and which constitute a part of this specification, illustrate certain embodiments of the invention, and together with the detailed description serve to explain the principles of the present invention.

### BRIEF DESCRIPTION OF THE DRAWINGS

In order to assist the understanding of this invention, reference will now be made to the appended drawings, in which like reference characters refer to like elements. The drawings are exemplary only, and should not be construed as limiting the invention.

FIG. 1 is a schematic view of a bifurcated prosthetic graft with an orifice according to an embodiment of the present invention;

FIG. 2 is a schematic view of a tubular prosthetic graft with a plurality of orifices according to another embodiment of the present invention;

FIG. 3 is a schematic of a patch prosthetic graft with a plurality of orifices according to another embodiment of the present invention;

FIG. 4 is a schematic view of a tubular prosthetic graft with an orifice including a plurality of radiopaque markers according to an embodiment of the present invention;

FIG. 5 is a schematic view of a tubular prosthetic graft with an orifice including a plurality of radiopaque markers and a prosthetic patch graft with an orifice including a plurality of radiopaque markers according to an embodiment of the present invention;

FIG. 6 is a schematic view of a tubular prosthetic graft used in conjunction with a unitary patch prosthetic graft assembly in accordance with an embodiment of the present invention;

FIG. 7 is a schematic view of a tubular prosthetic graft with a plurality of orifices and a plurality of radiopaque markers according to another embodiment of the present invention; and

FIG. 8 is a schematic view of the tubular prosthetic graft of FIG. 7 located in a vessel with branching arteries according to an embodiment of the present invention.

### DETAILED DESCRIPTION OF EMBODIMENTS OF THE INVENTION

The following descriptions of embodiments of the present invention are described, for purpose of example, in connection with the repair of an abdominal aortic aneurysm. The inventors of the present subject matter contemplate that the embodiments described herein are capable of use in the repair of other vessels and in other procedures. Thus, it is intended that the present invention cover the modifications and variations of the invention, provided they come within the scope of the appended claims and their equivalents.

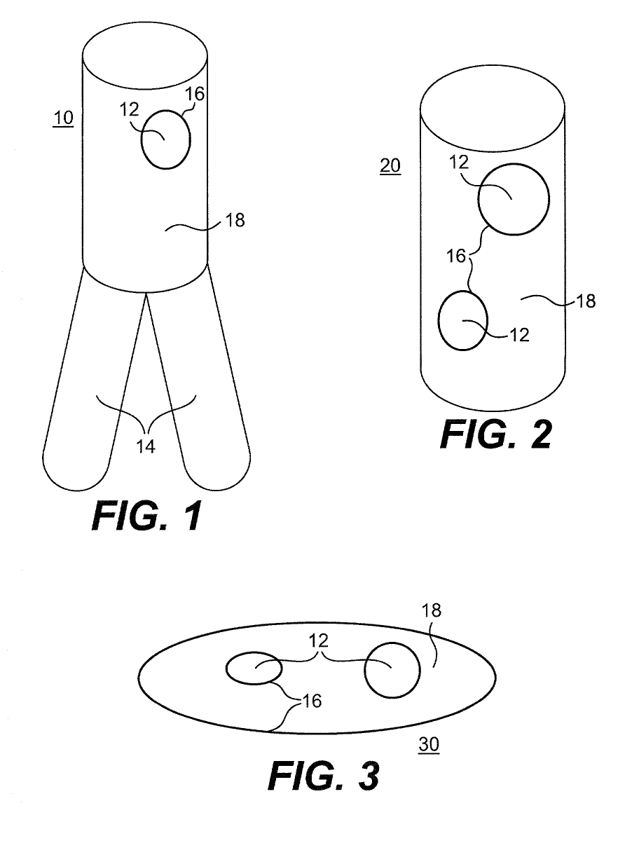

Reference will now be made in detail to embodiments of a surgical component, such as a prosthetic graft, according to the present invention for use in repairing a vessel during a surgical procedure, examples of which are illustrated in FIGS. 1-3.

FIGS. 1, 2, and 3 depict embodiments of a surgical component of the present invention directed to a bifurcated graft 10, a tubular graft 20, and a patch graft 30 for use in repairing a vessel during a surgical procedure. The bifurcated graft 10, the tubular graft 20, and the patch graft 30 may include an orifice 12 or a plurality of orifices 12. The body portion 18 of the prosthetic graft 10, 20, or 30 may be manufactured from a graft material suitable for surgical procedures wherein the body portion 18 may comprise Dacron®, polyester, polytetrafluroroethylene (PTFE), or any other suitable material.

In an embodiment of the present invention, the bifurcated graft 10 may comprise an elongated body 18 and one or more limbs 14. The elongated body 18 and the limbs 14 may be of any size, length, diameter, or shape complementary to the vessel for repair. The tubular graft 20 may also comprise an elongated body 18 of any size, shape, diameter or length complementary to the vessel for repair. The patch graft 30 may be of any size, shape, or length complementary to the vessel for repair.

In an alternative embodiment, the surgical component, the orifice 12, and/or the plurality of orifices 12 may be customized to a specific location on the surgical component. Customized means that the surgical component may be customized to the anatomy of the component recipient. The customization may include the length, diameter, size, shape, number of limbs (if any), size and position of orifice(s) (if any), location of the surgical component and/or orifice(s) in relation to the vessel to be repaired and/or any vessels branching from the vessel to be repaired. The body 18 and one or more limbs 14 may also be customized in relation to the vessel to be repaired. The surgical component may be modified at the time of insertion, or in an alternative embodiment of the present invention, the surgical component may be customized by the supplier based on the imaging of the component recipient. Imaging techniques may include techniques such as, but not limited to: sonography, duplex scanning, intravascular ultrasound (IVUS), magnetic resonance (MR), and/or radiologic imaging of any type including but not limited to angiography of any type, computer tomography (CT) of any type, or any other suitable imaging technique.

In an alternative embodiment, the bifurcated graft 10, the tubular graft 20, and the patch graft 30 may comprise a detectable indicator such that the shape, orientation, and location of the surgical component, or of the surgical component and the one or more orifices 12, are identifiable using any suitable detection means. Detection means may be any means capable of detecting the indicator. The indicator may comprise a plurality of radiopaque markers 16 yielding detectable generic markings such that the shape, location, and/or orientation of the surgical component and/or the orifice(s) is identifiable. Generic markings may include a repetitive pattern, bands of any width, or any type of marking leading to a distinction. Different generic markings may be used to identify different portions of the surgical component and/or the orifice(s). The radiopaque markers 16 may comprise any identifiable marker, including but not limited to, wire, vaporized metal, or impregnated radiopaque coatings wherein the radiopaque markers may be noble or heavy compositions or any other radiopaque metals, alloy, or materials, that may include, but are not limited to, the following: platinum, gold, tantalum, titanium, copper, molybdenum, barium-sulphite, tungsten, palladium, iridium, or rhodium.

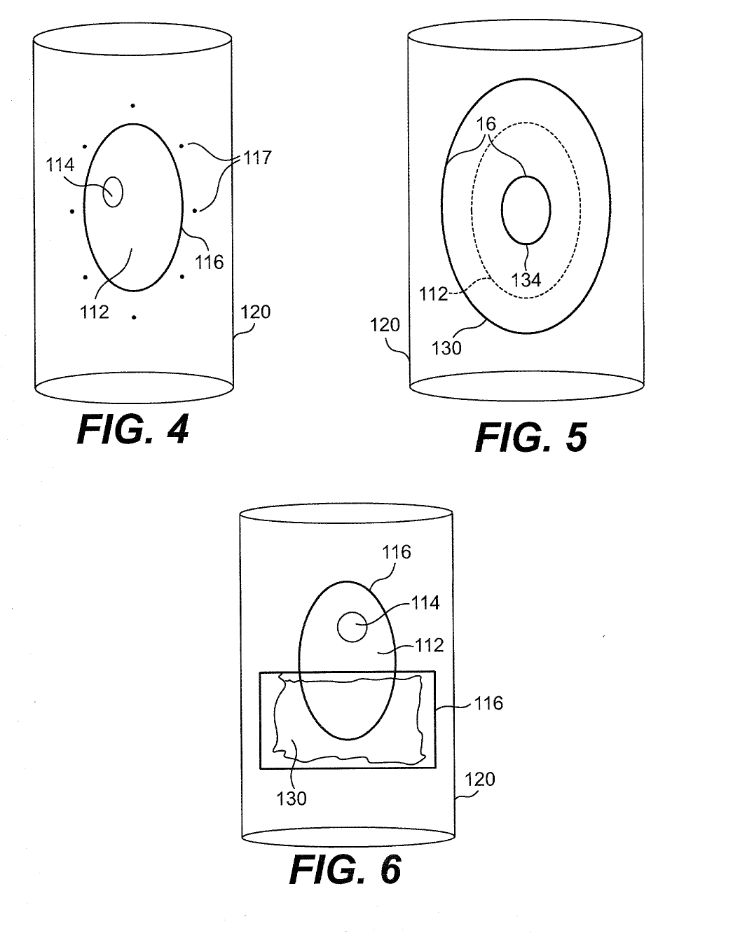

FIG. 4 illustrates an enlarged view of a tubular graft 120 with an orifice 112. The orifice 112 corresponds to the location of a branching vessel 114, which is branching from the vessel to be repaired (not shown). The tubular graft 120 and orifice 112 may or may not be customized to the location of the branching vessel at the site of repair. Exemplified also in FIG. 4 is the use of a plurality of radiopaque markers 116 for indicating the location of the orifice 112. Attachment means 117, such as, but not limited to, sutures, surgical fasteners, or any other suitable attachment means may be used around the orifice to attach the graft to the vessel to be repaired at the site of the branching vessel.

In an embodiment of the present invention as shown in FIG. 3, the prosthetic graft 30 comprises a body portion 18. The body portion 18 may be flat and may be of any shape or size. The body portion 18 may also be customized such that the orifice of the prosthetic graft corresponds to the location of a branching vessel or the orifice of another prosthetic graft. The prosthetic graft 30 may be used alone or in conjunction with another surgical component, including, but not limited to a tubular graft or a bifurcated graft. Another embodiment of the invention comprises a prosthetic graft 30 having an orifice 12 located on the body portion 18 wherein the orifice corresponds to the location of a vessel branching from the vessel to be repaired, or to an orifice in another graft, such that, when in the desired position within the vessel to be repaired, or the other graft, the orifice of the prosthetic graft 30 allows for the perfusion of blood into the branching vessel.

In an alternative embodiment, the prosthetic graft 30 may be used in conjunction with another surgical component and may comprise an indicator such that the shape, orientation, and location of the prosthetic graft 30 is identifiable using any suitable detection means. The indicator may comprise a plurality of radiopaque markers 16 yielding generic markings such that the orifice 12 of the prosthetic graft 10 or 20 and the orifice 12 of the prosthetic graft 30 may be detected. In another alternative embodiment of the present invention, generic markings may be used such that the orifice 12 of the prosthetic graft 10 or 20 and the orifice 12 of the prosthetic graft 30 may be differentiated. Generic markings may also be added to help in the determination of the location and orientation of the grafts.

FIG. 5 is an enlarged view of the tubular graft 120 of FIG. 4 used in conjunction with a prosthetic graft 130. Prosthetic graft 130 has a smaller orifice 134 when compared to the orifice 112 of graft 120. Prosthetic graft 130 may be customized for the size of the orifice required to allow for blood flow into the branching vessel, while also being small enough to provide support and help protect the area of the vessel to be repaired from stress in the region where the branching vessel meets the vessel to be repaired. In an alternative embodiment, the prosthetic graft 130 may be radiopaquely labeled 16.

In an alternative embodiment, a prosthetic graft 130 without an orifice and a prosthetic graft 10, 20, or 120 with at least one orifice may be used individually or in combination as shown in FIG. 6. The use of the prosthetic graft 130 without an orifice is shown. A tubular graft 120 (from FIG. 4) is reconstructed to allow for the continuous blood flow with the addition of a prosthetic graft 130. The prosthetic graft 130 is placed over the orifice 112 in graft 120 having a branching vessel 114. In another alternative embodiment of the present invention, the orifice 112 and the prosthetic graft 130 are identified by radiopaque markers 116.

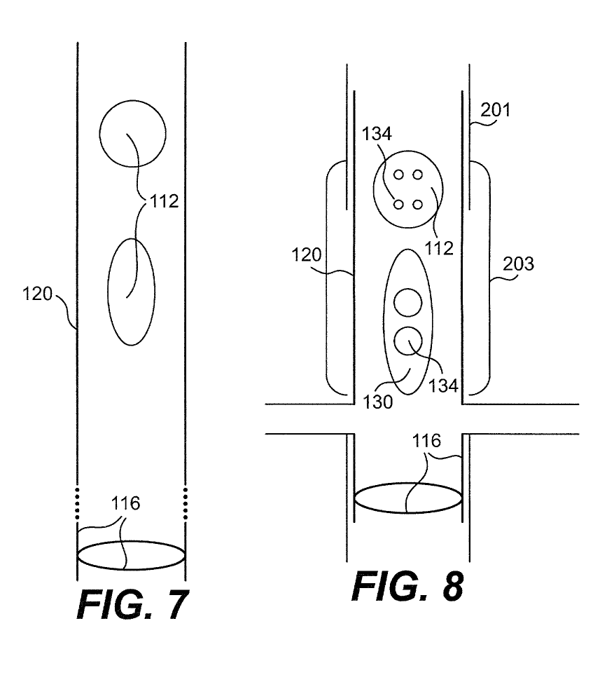

In another embodiment of the invention illustrated in FIG. 8, a thoracic aorta 201 with an aneurysm 203 is restored by a tubular graft 120. The tubular graft 120 prior to insertion in the thoracic aorta 201 may be customized by any suitable imaging technique yielding an image dependent on the size, location, and shape of the vessel to be repaired and/or the size, location, and shape of any branching vessels. The resulting tubular graft 120 with orifices 112 is illustrated by FIG. 7. The restored thoracic aorta 201 in FIG. 8 demonstrates a plurality of orifices of prosthetic graft 120 aligned with the branching vessels 134. The prosthetic graft 120 has a varying number of orifices 112 to accommodate any number of branching vessel attachments.

It will be apparent to those skilled in the art that various modifications and variations can be made in the construction and configuration of the present invention, without departing from the scope or spirit of the invention. For instance, such surgical components can be used in any blood vessel. A common application may include a tubular graft with numerous holes specifically placed to allow perfusion of blood vessels to or from: the spinal cord; the intestines; the kidneys; the brain; the pelvis; and/or the extremities. Specifically, as depicted in FIG. 8 of a thoracoabdominal aortic aneurysm involving the intercostal arteries supplying the spinal cord, involving the mesenteric vessels (celiac and superior mesenteric arteries) and involving the renal arteries, a surgical component can be prepared, based on imaging data regarding a specific patient's anatomy, by placing multiple orifices outlined with radiopaque markers in a tubular graft. These orifices can be placed such that arterial blood flow can continue to be supplied to the spinal cord, the intestines, the kidneys, and any other appropriate organs.

The surgical component may be inserted into the aorta through a standard or specialized delivery apparatus, including, but not limited to, an introducer sheath, and placed into position. The surgical component may then be held in place by any of a variety of support methods and may be secured to the vessel in any order by any suitable means.

This approach might allow endovascular repair of thoracoabdominal aortic aneurysms with constant perfusion of the arteries to the spinal cord, intestines, and kidneys. If temporary occlusion of the aorta or any branch vessel is necessary, it is likely that, with this approach, the occlusion time would be significantly shortened over the occlusion time obligatorily required during an open procedure.

The patch prosthetic graft 30, with no or one or more orifices can be used in any vessel for any purpose. Examples of possible uses could be to reinforce a vessel dissection, a traumatic injury of a blood vessel, or a combination of a vessel and a previously inserted surgical component. A tubular prosthetic graft 20 could be used for these purposes as well. Specifically, it may be advantageous to create an overly large orifice in a tubular graft in order to facilitate placement of the graft without jeopardizing the orifice of an intended or critical vessel. Once the entire tubular graft has been secured, it may be desirable to effectively reduce the size of the orifice over the intended branching vessel site.

The surgical components may be further adapted to provide orifices, ringed or not by radiopaque markings, that correspond to any combination of arteries, including, but not limited to: intercostals; lumbars; superior mesenteric; celiac; or renals. A range of products may be created that combine surgical components and radiopaque markings in a number of unique, procedure-benefiting configurations.

Radiopaque markings may be applied by vacuum-deposit, screen-printing, or any other suitable techniques. They may also be applied by standard techniques of placement of radiopaque wires, with a unique feature comprising holes in the sides of prosthetic grafts, which may be made radiopaque by markers.

Examples 1-4 of the present invention are exemplary and illustrative only and are not intended to limit the invention as claimed to any particular embodiment or combination of embodiments.

### EXAMPLE 1

In this proximal collar concept, a band of radiopaque material is deposited around the proximal neck of a prosthetic graft. The band is comprised of a series of equi-spaced, identically-similar slanted lines of a width and coating thickness that enables their easy detection when under radiological purview. The slanted lines enable distinguishment of a forward segment of graft from an aft segment. Differentiation is not necessary, but represents an embodiment of the present invention. Other repetitive patterns may be utilized, such as, for example, a series of chevrons, providing similar visualization benefits. A dogtooth pattern may enable both the accurate, sequential placement of fastening means, such as, for example, sutures or fasteners, within the graft/tissue matrix and the preservation of tissue integrity.

### EXAMPLE 2

In this distal collar concept, a band of radiopaque material is deposited around the distal necks of each of the two limbs of a bifurcated prosthetic graft. The band of this example is similar in every respect to the band of Example 1, but additionally provides a docking feature that enables the accurate and appropriate attachment therein or about of secondary stented limbs, also banded, which extend there from and are fastened into other vessels, such as, but not limited to, the iliac arteries. When correctly positioned with respect to one another, the overlapping limbs will present, under radiological view, as a series of crosshatched lines. Again, differentiation is not necessary, but merely represents an embodiment of the present invention.

### EXAMPLE 3

In this midsection collar concept, a band of radiopaque material is deposited around the midsection of a prosthetic graft extending from the band of the proximal neck to its distal termination (banded or not). The band of this product is comprised of a series of equi-spaced, identically similar lines of a width and coating thickness that enables their easy detection when under radiological purview. The incorporated line type provides an indication of both flow direction, proximal-to-distal, and therefore proof of correct graft deployment and graft kinking. If the graft is kinked, the rigid pattern of lines will appear disrupted when viewed radiologically.

### EXAMPLE 4

In this virtual stent concept, a radiopaque band or bands of any width are uniformly disposed over either the inner or outer prosthetic graft surface. The band is composed of a pattern of repetitively positioned fenestrations having a coating thickness that facilitates the unencumbered delivery of the graft to the surgical site within the descending aorta. The coating is of such a material and thickness, however, that upon the graft's subsequent expansion within the aortic lumen, the individual fenestrations open up to form a “chain mail”-like configuration, having an appreciable structural integrity. Not dissimilar to a stent, the coated graft is absent the attendant shortcomings of that product. There are neither attachment problems of graft to stent, nor friction/abrasion problems between graft and stent materials as a consequence of the complete assimilation of the coating within the graft fabric. The performance features of Examples 1-3 may be integrated into this example.

It will be apparent to those skilled in the art that various modifications and variations can be made in the construction and configuration of the present invention, without departing from the scope or spirit of the invention. It is intended that the present invention cover the modifications and variations of the invention, provided they come within the scope of the appended claims and their equivalents.

What is claimed is:

1. A surgical component for use during a surgical procedure, said surgical component comprising:
an elongated body having an open proximal end and an open distal end, wherein said body further comprises at least one orifice located between said proximal end and said distal end.

2. The surgical component according to claim 1, wherein said elongated body is customized to a surgical patient.

3. The surgical component according to claim 1, wherein said at least one orifice is customized for use during the surgical procedure.

4. The surgical component according to claim 1, further comprising an indicator complementary to said at least one orifice, wherein said at least one orifice is delineated by said indicator.

5. The surgical component according to claim 4, wherein said indicator comprises a plurality of radiopaque markers.

6. The surgical component according to claim 1, further comprising an indicator complementary to said elongated body, wherein said elongated body is delineated by said indicator.

7. The surgical component according to claim 6, wherein said indicator comprises a plurality of radiopaque markers.

8. A surgical component for use during a surgical procedure, said surgical component comprising:
an elongated body having an open proximal end and an open distal end and one or more limbs attached thereto, wherein said body further comprises at least one orifice located between said proximal end and said distal end.

9. The surgical component according to claim 8, wherein said elongated body is customized to a surgical patient.

10. The surgical component according to claim 8, wherein said at least one orifice is customized for use during the surgical procedure.

11. The surgical component according to claim 8, further comprising an indicator complementary to said at least one orifice, wherein said at least one orifice is delineated by said indicator.

12. The surgical component according to claim 11, wherein said indicator comprises a plurality of radiopaque markers.

13. The surgical component according to claim 8, further comprising an indicator complementary to said elongated body and said one or more limbs, wherein said body and said limbs are delineated by said indicator.

14. The surgical component according to claim 13, wherein said indicator comprises a plurality of radiopaque markers.

15. A surgical component for use during a surgical procedure, said surgical component comprising a body portion having at least one orifice located thereon.

16. The surgical component according to claim 15, wherein said body portion is flat.

17. The surgical component according to claim 15, wherein said body portion is customized for use during the surgical procedure.

18. The surgical component according to claim 15, wherein said at least one orifice is customized for use during the surgical procedure.

19. The surgical component according to claim 15, further comprising an indicator complementary to said at least one orifice, wherein said indicator delineates said at least one orifice.

20. The surgical component according to claim 19, wherein said indicator comprises a plurality of radiopaque markers.

21. The surgical component according to claim 15, further comprising an indicator complementary to said body portion, wherein said indicator delineates said body portion.

22. The surgical component according to claim 21, wherein said indicator comprises a plurality of radiopaque markers.

23. A surgical component assembly for use during a surgical procedure, said surgical component assembly comprising:
a first surgical component comprising an elongated body with an open proximal end and an open distal end, wherein said first surgical component further comprises at least one orifice located between said proximal end and said distal end; and
a second surgical component comprising a body portion adapted for placement over at least a portion of said at least one orifice of said first surgical component.

24. The surgical component assembly according to claim 23, wherein said body portion of said second surgical component further comprises at least one orifice located thereon.

25. The surgical component assembly according to claim 24, wherein said at least one orifice of said second surgical component is of a smaller diameter than said at least one orifice of said first surgical component.

26. The surgical component assembly according to claim 24, further comprising a first indicator complementary to said at least one orifice of said first surgical component and a second indicator complementary to said at least one orifice of said second surgical component for delineating said orifices.

27. The surgical component assembly according to claim 26, wherein said first indicator and said second indicator are the same.

28. The surgical component assembly according to claim 26, wherein said first indicator and said second indicator are different.

29. The surgical component assembly according to claim 23, further comprising a first indicator complementary to said elongated body of said first surgical component and a second indicator complementary to said body portion of said second surgical component for delineating said first surgical component and said second surgical component.

30. The surgical component assembly according to claim 23, wherein said first surgical component further comprises one or more limbs attached to said elongated body.

31. The surgical component assembly according to claim 30, wherein said body portion of said second surgical component further comprises at least one orifice located thereon.

32. The surgical component assembly according to claim 31, wherein said at least one orifice of said second surgical component is of a smaller diameter than said at least one orifice of said first surgical component.

33. The surgical component assembly according to claim 31, further comprising a first indicator complementary to said at least one orifice of said first surgical component and a second indicator complementary to said at least one orifice of said second surgical component for delineating said orifices.

34. The surgical component assembly according to claim 33, wherein said first indicator and said second indicator are the same.

35. The surgical component assembly according to claim 33, wherein said first indicator and said second indicator are different.

36. The surgical component assembly according to claim 30, further comprising a first indicator complementary to said elongated body and said one or more limbs of said first surgical component and a second indicator complementary to said body portion of said second surgical component for delineating said first surgical component and said second surgical component.

37. A method of customizing a surgical component for use in a surgical patient, comprising the step of:
adjusting the size of the surgical component to conform to the surgical patient.

38. The method according to claim 37, further comprising one or more of the steps of:
adjusting the position of at least one orifice located on the surgical component;
adjusting the shape of at least one orifice located on the surgical component; and
adjusting the number of orifices located on the surgical component.

39. A method of labeling a surgical component having at least one orifice for use in a surgical procedure, comprising the step of:
radiopaquing the at least one orifice such that the at least one orifice is identifiable.

40. A method of labeling a surgical component for use in a surgical procedure, comprising the step of:
radiopaquing a body portion of the surgical component such that the surgical component is identifiable.

41. A method of repairing a vessel at a surgical site, comprising the steps of:
delivering a first surgical component having at least one orifice thereon to the surgical site;
positioning the at least one orifice of the first surgical component to correspond to at least one branching vessel; and
positioning a second surgical component over at least a portion of the least one orifice of the first surgical component.
